(19) United States
(12) Patent Application Publication
(10) Pub. No.: US 2004/0038127 A1
(43) Pub. Date: Feb. 26, 2004

(54) Small cation/delocalizing anion as an ambient temperature molten salt in electrochemical power sources

(76) Inventor: Carl Roger Schlaikjer, Concord, MA (US)

Correspondence Address: WILSON GREATBATCH TECHNOLOGIES, INC., 10,000 WEHRLE DRIVE, CLARENCE, NY, 14031, US

(21) Appl. No.: 10/642,045

(22) Filed: Aug. 15, 2003

Related U.S. Application Data
(60) Provisional application No. 60/404,813, filed on Aug. 20, 2002.

(51) Int. Cl. H01M 6/14; H01M 10/40; C07F 5/02; C07C 211/62

(52) U.S. Cl. 429/199; 429/303; 252/62.2; 568/5; 564/8

(57) ABSTRACT

The present invention is directed to the use of a new ambient temperature molten salt as an electrolyte for electrochemical energy storage devices, such as electrochemical cells and electrolytic capacitors. The ambient temperature molten salt comprises an imide cation combined with a small anion. A particularly preferred anion is bis-trifluoromethanesulfonyl imide. The electrolyte is useful with electrochemical devices such as primary and secondary electrochemical cells and capacitors of the electrolytic and electrolytic/electrochemical hybrid types.

### CROSS REFERENCE TO RELATED APPLICATION

The present invention claims priority to U.S. provisional application Serial No. 60/404,813, filed Aug. 20, 2002.

### BACKGROUND OF THE INVENTION

1. Field of the Invention

This invention relates to electrochemical power sources such as cells, batteries and capacitors. More particularly, the present invention is directed to small cations and delocalizing anions that form into molten salts useful as electrolytes in primary and secondary electrochemical cells and in high energy density electrolytic capacitors. Additionally, such salts are useful as hydraulic fluids and fire retardants.

2. Prior Art

Examples of electrolytes currently being used in rechargeable electrochemical power sources include liquid, gel, and dry polymer types. Dry polymer electrolyte cells without plasticizers exist, but their inadequate conductivity and low lithium ion transference prevent them from being used at ambient or reduced temperatures.

Liquid and gel electrolytes have higher ionic conductivity and adequate lithium ion transference when compared with dry polymer electrolytes. An example is a solvent system of propylene carbonate and 1,2-dimethoxyethane having a lithium salt such as LiPF₆ or LiAsF₆ dissolved therein. Such an electrolyte is typically used to activate a lithium/silver vanadium oxide (Li/SVO) cell. Additionally, liquid and gel electrolyte cells, such as of a carbonaceous negative electrode and a lithium cobalt oxide positive electrode, are capable of cycling at relatively high rates and low temperatures. One major disadvantage with them, however, is that organic solvents must be included in the electrolyte to improve conductivity and, in the case of the liquid phase, lower viscosity. Liquid and gel electrolytes are also relatively volatile and flammable, which poses a risk of fire when they are heated. In addition, liquid and gel electrolyte cells, whether of a primary or a secondary chemistry, are subject to gassing and subsequent leakage. The packaging and processing required to prevent leakage is complex and, therefore, costly. In contrast, electrolytes based on ambient temperature molten salts promise the safety of dry polymers along with substantially higher ionic conductivities.

The prior art describes electrochemical power sources having electrolytes containing bis-trifluoromethanesulfonyl imide anions. For example, U.S. Pat. No. 5,652,072 to Lamanna et al. discloses that lithium bis-trifluoromethanesulfonyl imide, Li⁺(CF₃SO₂)₂N⁻, is a known electrolytic salt used with electrochemical cells. At col. 1, lines 19 to 23, this patent states that lithium bis-trifluoromethanesulfonyl imide “has good conductivity and stability, but is highly corrosive toward aluminum at potentials above 3V (vs Li/Li+).” In fact, lithium bis-trifluoromethanesulfonyl imide is so corrosive, it is discouraged from being used in most advanced, high voltage cells.

From that fundamental understanding, Lamanna et al. attempted to find variations of lithium bis-trifluoromethanesulfonyl imide that are not as deleterious. U.S. Pat. No. 6,280,883 to Lamanna et al. discloses at col. 2, line 60 to col. 3, line 16 a conductive salt having the formula of:

trialkylammonium⁺((R_{f1}SO₂)(R_{f2}SO₂)N)⁻

wherein R_{f1} and R_{f2} are each independently a straight or branched perfluoroalkyl group of 1 to 4 carbon atoms, with R_{f1} and R_{f2} having a total of up to 5 carbon atoms.

In effect, Lamanna et al. implicitly disclose that triethylammonium bis-trifluoromethanesulfonyl imide is a conductive salt useful with lithium ion batteries. This conductive salt is a solid, however, and must be combined with a surfactant salt similar to the above-identified conductive salt, but with longer R_{f1} and R_{f2} chains.

At col. 9, lines 38 to 45 of the '883 patent, Lamanna et al. confirm that there is only one type of conductive salt that does not need to be combined with a conductive surfactant when used in electrical power sources. This is an ionic liquid electrolyte, i.e., a molten salt, “which are inherently liquid at ambient temperature, e.g., 20 degrees Celsius or higher.”

Such molten salts are disclosed in U.S. Pat. No. 5,827,602 to Koch et al. This patent discloses that a preferred molten salt contains a cation and an anion as follow:

| | |
|---|---|
| Cation: | perfluoro-1-ethyl-3-methylimidazolium |
| Anion: | bis (trifluoromethanesulfonyl) imide |

At col. 3, line 56 to col. 4, line 16 of their patent, Koch et al. state that “it is believed that one of the causes of the desirable hydrophobic property of the ionic liquids [molten salt] is the large size of the cations and anions involved.” Accordingly, Koch et al. clearly teach away from using small cations for molten salt compositions in electrochemical power sources.

In addition, Koch et al. admit that their molten salts of relatively large cations and relatively large anions have poor ionic conductivity. Therefore, at col. 5, lines 10 to 15 of their patent they suggest that molten salts of large cations and large anions be used with polar organic liquids.

In that light, the present invention solves Koch et al.'s ionic conductivity problem without having to use a solvent in the molten salt.

### SUMMARY OF THE INVENTION

The present invention is directed to the use of a new ambient temperature molten salt as an electrolyte for electrochemical energy storage devices, such as electrochemical cells and electrolytic capacitors. The ambient temperature molten salt comprises a relatively small cation and a delocalizing anion with substituent organic groups. In order to increase resistance to electrochemical oxidation and reduction, the substituent organic groups are preferably halogenated, such as by fluorine. Preferred anions include bis-trifluoromethanesulfonyl imide and bis-pentafluoroethanesulfonyl imide.

The molten salt is used in its liquid form, or is combined with a polymer to provide a gel electrolyte. Either type of nonaqueous electrolyte provides high conductivity in an electrochemical system without the use of volatile components. There is also a significant decrease in risk of fire if the cell or capacitor is overheated or overcharged, even in the absence of safety circuits. This improved safety is without loss in capacity, cycle life, or rate capability relative to the existing technology, such as the above-discussed Koch et al. electrolytes. Cells and capacitors of the present invention are also easier to manufacture and to package than those activated with conventional electrolytes.

These and other objects of the present invention will become increasingly more apparent to those skilled in the art by reference to the following description.

### DETAILED DESCRIPTION OF THE PREFERRED EMBODIMENTS

The present invention is directed to a unique molten salt composition having a relatively small cation and an anion. The anion provides extensive delocalization of the negative charge. Examples of such anions include, but are not limited to closocarborates, for example B₉H₉CH⁻, B₁₁H₁₁CH⁻, and halogenated derivatives thereof, closoborates, for example B₁₀H₁₀²⁻, B₁₂H₁₂ ²⁻, and halogenated derivatives thereof, triflate (CF₃SO₃⁻), ClO₄⁻, C(SO₂CF₃)₃⁻, N(SO₂CF₃)₂⁻, O₃SCF₃⁻, C₆F₅SO₃⁻, O₂CCF₃⁻, and mixtures thereof; and anions of the following formula:

((R_{f1}SO₂)(R_{f2}SO₂)N)⁻

wherein R_{f1} and R_{f2} are each independently a straight or a branched perhalogenated alkyl group of 1 to 4 carbon atoms, with R_{f1} and R_{f2} having up to 5 carbon atoms. The preferred halogen is fluorine. Preferred anions are bis-trifluoromethanesulfonyl imide and bis-pentafluoroethanesulfonyl imide.

The bis-trifluoromethanesulfonyl imide anion is capable of assuming five resonant hybrid structures, as indicated below.

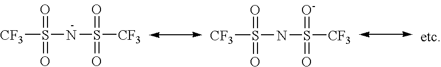

bis-trifluoromethanesulfonyl imide

Asymmetric derivatives of bis-trifluoromethanesulfonyl imide, such as trifluoromethanesulfonyltrifluoroacetyl imide and trifluoromethanesulfonylpentafluoroethanesulfonyl imide, are also useful as anions.

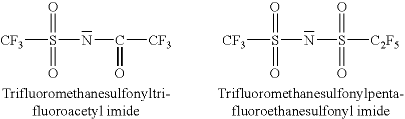

The cation of the present invention must be relatively small. Examples of small cations include, but are not limited to, nitrogen onium cations such as ammonium, dialkylammonium, trialkylammonium, and tetralkylammonium, wherein the alkyl has 1 to 4 carbon atoms and can be partially or totally halogenated. Halogenated alkyl groups include fully or partially halogenated ethyl, propyl, isopropyl, butyl, isobutyl, sec-butyl, and tert-butyl groups. Halogens include fluorine, chlorine, and bromine. Preferred nitrogen onium cations are triethylammonium and trimethylammonium cations.

The small cation/delocalizing anion molten salt product, for example, but not limited to triethylammonium bis-trifluoromethanesulfonyl imide, is liquid at ambient temperature and only slightly soluble in water. Being liquid at ambient temperature means that the electrolyte is in a liquid phase at a temperature of about 60° C., or less.

One convenient method of preparing this product compound is by reacting two aqueous salt solutions, one containing triethyl amine with a stoichiometric amount of hydrochloric acid, and the other containing lithium bis-trifluoromethanesulfonyl imide. The slightly soluble product triethylammonium bis-trifluoromethanesulfonyl imide separates as a heavier liquid phase and may be drawn off, for example, by using a separatory funnel. The product may be washed one or more times by equilibration with deionized water, then dried in vacuo while being heated.

If a single-phase gel electrolyte is preferred, the molten salt is mixed with an unsaturated monomer. Suitable polymerizerable monomers have at least one α-unsaturated functionality, and more preferably multiple α-unsaturated functionalities, such as multi-functional (meth)acrylates so that they are relatively rapidly curable inside a cell casing to form a cross-linked matrix or network. Preferably, the (methyl)acryloyl monomer has at least one functional group selected from the group consisting of alkyl, alkyl ether, alkoxylated alkyl and alkoxylated phenol functional groups. Suitable monomers include dipentaerythritol hexaacrylate (DPHA), dipentaerythritol pentaacrylate (DPAA), pentaerythritol tetraacrylate, ethoxylated pentaerythritol tetraacrylate, di(trimethylolpropane) tetraacrylate (DTMPTA), trimethylolpropane trimethacrylate, ethoxylated trimethylolpropane triacrylate (ETMPTA), ethoxylated bisphenol diacrylate, hexanediol diacrylate, and mixtures thereof. For more detail regarding gel electrolytes, reference is drawn to U.S. application Ser. No. 10/000,883, filed Nov. 15, 2001. This application is assigned to the assignee of the present invention and incorporated herein by reference.

The present ambient temperature molten salts are useful as electrolytes in a wide variety of electrochemical power sources. These include primary electrochemical cells, such as of the lithium/silver vanadium oxide (Li/SVO), lithium/copper silver vanadium oxide (Li/CSVO), and lithium/manganese oxide (Li/MnO₂) couples. Exemplary Li/SVO cells are described in U.S. Pat. Nos. 4,310,609 and 4,391,729, both to Liang et al., and 5,580,859 to Takeuchi et al. while an exemplary Li/CSVO cell is described in U.S. Pat. Nos. 5,472,810 and 5,516,340, both to Takeuchi et al. All of these patents are assigned to the assignee of the present invention and incorporated herein by reference.

The ambient temperature molten salts of the present invention are also useful for activating secondary electrochemical cells. In a secondary system, the negative electrode comprises a material capable of intercalating and de-intercalating the active material, such as the preferred alkali metal lithium. A carbonaceous negative electrode comprising any of the various forms of carbon (e.g., coke, graphite, acetylene black, carbon black, glassy carbon, “hairy carbon” etc.) that are capable of reversibly retaining the lithium species is preferred for the negative electrode material. A “hairy carbon” material is particularly preferred due to its relatively high lithium-retention capacity. “Hairy carbon” is a material described in U.S. Pat. No. 5,443,928 to Takeuchi et al., which is assigned to the assignee of the present invention and incorporated herein by reference. Graphite is another preferred material. Regardless of the form of the carbon, fibers of the carbonaceous material are particularly advantageous because they have excellent mechanical properties that permit them to be fabricated into rigid electrodes capable of withstanding degradation during repeated charge/discharge cycling. Moreover, the high surface area of carbon fibers allows for rapid charge/discharge rates.

Also in secondary systems, the positive electrode preferably comprises a lithiated material that is stable in air and readily handled. Examples of such air-stable lithiated cathode active materials include oxides, sulfides, selenides, and tellurides of such metals as vanadium, titanium, chromium, copper, molybdenum, niobium, iron, nickel, cobalt and manganese. The more preferred oxides include LiNiO₂, LiMn₂O₄, LiCoO₂, LiCO_{0.92}Sn_{0.08}O₂ and LiCo₁₋ₓNiₓO₂.

The present ambient temperature molten salts are not only useful as electrolytes in primary and secondary electrochemical cells, they are useful in capacitors as well. This includes conventional electrolytic capacitors, as well as those of an electrolytic/electrochemical hybrid type. Capacitor cathodes commonly used in electrolytic capacitors include etched aluminum foil in aluminum electrolytic capacitors, and those commonly used in wet tantalum capacitors such as of silver, sintered valve metal powders, platinum black, and carbon. The cathode of hybrid capacitors include a pseudocapacitive coating of a transition metal oxide, nitride, carbide or carbon nitride, the transition metal being selected from the group consisting of ruthenium, cobalt, manganese, molybdenum, tungsten, tantalum, iron, niobium, iridium, titanium, zirconium, hafnium, rhodium, vanadium, osmium, palladium, platinum, and nickel. The pseudocapacitive coating is deposited on a conductive substrate such as of titanium or tantalum. The electrolytic/electrochemical hybrid capacitor has high energy density and is particularly useful for implantable medical devices such as a cardiac defibrillator.

The anode is of a valve metal consisting of the group vanadium, niobium, tantalum, aluminum, titanium, zirconium and hafnium. The anode can be a foil, etched foil, sintered powder, or any other form of porous substrate of these metals.

A preferred chemistry for a hybrid capacitor comprises a cathode electrode of a porous ruthenium oxide film provided on a titanium substrate coupled with an anode of a sintered tantalum powder pressed into a pellet. A suitable separator material impregnated with the present working electrolyte segregates the cathode and anode electrodes from each other. Such a capacitor is described in U.S. Pat. Nos. 5,894,403 to Shah et al., U.S. Pat. No. 5,920,455 to Shah et al. and U.S. Pat. No. 5,926,362 to Muffoletto et al. These patents are assigned to the assignee of the present invention and incorporated herein by reference.

It has been found that the small cation/delocalizing anion molten salt of the present invention is less viscous and therefore more conductive than other molten salts, and does not require additional solvents for its corresponding solid salt compositions. Notwithstanding those observations, one would expect the instant invention to react with lithium metal. However, no such reaction has been observed. In addition, triethylammonium bis-trifluoroethanesulfonyl imide is a good solvent for lithium bis-trifluoromethanesulfonyl imide.

The following example describes the preparation of an ambient temperature salt according to the present invention, and it sets forth the best mode contemplated by the inventors of carrying out the invention, but it is not to be construed as limiting.

### EXAMPLE I

Triethylammonium bis-trifluoromethanesulfonyl imide was prepared as follows: 25.8 grams (90 mm) of Li⁺(CF₃SO₂)₂N⁻ were dissolved in about 30 ml of water in a 200 ml beaker and transferred to a 125 ml separatory funnel. About 8 ml of 12 M hydrochloric acid (96 mm) were added with stirring to about 30 ml of water in a 200 ml beaker. To this solution was then slowly added 13.9 ml (90 mm) of triethyl amine, with stirring. The solution was then added to the separatory funnel and agitated. The mixture was allowed to separate, and the denser molten salt was drawn off the bottom. It was washed three times in the separatory funnel with 50 ml portions of water, and then dried in vacuo at 110° C. for 12 hours. Yield was 26 grams (77%) of a crystal clear, colorless liquid.

It is appreciated that various modifications to the inventive concepts described herein may be apparent to those of ordinary skill in the art without departing from the scope of the present invention as defined by the herein appended claims.

What is claimed is:

1. An ambient temperature molten salt as an electrolyte, which comprises:
a) a small nitrogen onium cation; and
b) an anion selected from the group consisting of closocarborates and halogenated derivatives thereof, closoborates and halogenated derivatives thereof, CF₃SO₃⁻, ClO₄⁻, C(SO₂CF₃)₃⁻, N(SO₂CF₃)₂⁻, O₃SCF₃⁻, C₆F₅SO₃⁻, O₂CCF₃⁻, and those of the following formula:
((R_{f1}SO₂)(R_{f2}SO₂)N)⁻
wherein R_{f1} and R_{f2} are each independently a straight or a branched perhalogenated alkyl group of 1 to 4 carbon atoms, with R_{f1} and R_{f2} having up to 5 carbon atoms, and mixtures thereof.

2. The electrolyte of claim 1 wherein the small nitrogen onium cation is selected from the group consisting of ammonium, dialkylammonium, trialkylammonium, and tetralkylammonium, wherein the alkyl has 1 to 4 carbon atoms and can be partially or totally halogenated.

3. The electrolyte of claim 2 wherein the alkyl is selected from the group consisting of methyl, ethyl, propyl, isopropyl, butyl, isobutyl, sec-butyl, and tert-butyl.

4. The electrolyte of claim 2 wherein the alkyl are halogenated alkyl groups selected from methyl, ethyl, propyl, isopropyl, butyl, isobutyl, sec-butyl, and tert-butyl.

5. The electrolyte of claim 4 wherein the halogenated alkyl groups are at least partially halogenated.

6. The electrolyte of claim 4 wherein the halogen is selected from the group consisting of fluorine, chlorine, bromine, and mixtures thereof.

7. The electrolyte of claim 1 wherein the anion is selected from the group consisting of bis-trifluoromethanesulfonyl imide, trifluoromethanesulfonyltrifluoroacetyl imide and trifluoromethanesulfonylpentafluoroethanesulfonyl imide, and mixtures thereof.

8. The electrolyte of claim 1 wherein the cation is triethylammonium or trimethylammonium and the anion is bis-trifluoromethanesulfonyl imide or bis-pentafluoroethanesulfonyl imide.

9. The electrolyte of claim 1 is in a liquid phase at about 60° C., or less.

10. The electrolyte of claim 1 wherein the electrolyte is a gel electrolyte.

11. The electrolyte of claim 10 wherein the gel electrolyte includes an unsaturated monomer selected from the group consisting of dipentaerythritol hexaacrylate (DPHA), dipentaerythritol pentaacrylate (DPAA), pentaerythritol tetraacrylate, ethoxylated pentaerythritol tetraacrylate, di(trimethylolpropane) tetraacrylate (DTMPTA), trimethylolpropane trimethacrylate, ethoxylated trimethylolpropane triacrylate (ETMPTA), ethoxylated bisphenol diacrylate, hexanediol diacrylate, and mixtures thereof.

12. An electrochemical cell, which comprises:
a) a negative electrode of either lithium or having a material capable of intercalating and de-intercalating lithium;
b) a positive electrode comprising a cathode active material capable of intercalating lithium ions or capable of intercalating and de-intercalating lithium ions;
c) a separator disposed between the negative and positive electrodes to prevent direct physical contact between them;
d) an electrolyte activating the negative and the positive electrode, the electrolyte comprising:
i) a nitrogen onium cation; and
ii) an anion selected from the group consisting of closocarborates and halogenated derivatives thereof, closoborates and halogenated derivatives thereof, CF₃SO₃⁻, ClO₄⁻, C(SO₂CF₃)₃⁻, N(SO₂CF₃)₂⁻, O₃SCF₃⁻, C₆F₅SO₃⁻, O₂CCF₃⁻, and those of the following formula:
((R_{f1}SO₂)(R_{f2}SO₂)N)⁻
wherein Rf₁ and Rf2 are each independently a straight or branched perhalogenated alkyl group of 1 to 4 carbon atoms, with R_{f1} and R_{f2} having up to 5 carbon atoms, and mixtures thereof; and
e) a casing housing the negative and positive electrodes activated by the electrolyte.

13. The electrochemical cell of claim 12 wherein the small nitrogen onium cation is selected from the group consisting of ammonium, dialkylammonium, trialkylammonium, and tetralkylammonium and wherein the alkyl groups have 1 to 4 carbon atoms and may be partially or totally halogenated.

14. The electrochemical cell of claim 12 wherein the alkyl is selected from the group consisting of methyl, ethyl, propyl, isopropyl, butyl, isobutyl, sec-butyl, and tert-butyl.

15. The electrochemical cell of claim 12 wherein the halogenated alkyl groups are at least partially halogenated.

16. The electrochemical cell of claim 12 wherein the halogen is selected from the group consisting of fluorine, chlorine, bromine, and mixtures thereof.

17. The electrochemical cell of claim 12 wherein the anion is selected from the group consisting of bis-trifluoromethanesulfonyl imide, trifluoromethanesulfonyltrifluoroacetyl imide and trifluoromethanesulfonylpentafluoroethanesulfonyl imide, and mixtures thereof.

18. The electrochemical cell of claim 12 is in a liquid phase at about 60° C., or less.

19. The electrochemical cell of claim 12 wherein the cation is triethylammonium or trimethylammonium and the anion is bis-trifluoromethanesulfonyl imide or bis-pentafluoroethanesulfonyl imide.

20. The electrochemical cell of claim 12 wherein the electrolyte is a gel electrolyte.

21. The electrochemical cell of claim 20 wherein the gel electrolyte includes an unsaturated monomer selected from the group consisting of dipentaerythritol hexaacrylate (DPHA), dipentaerythritol pentaacrylate (DPAA), pentaerythritol tetraacrylate, ethoxylated pentaerythritol tetraacrylate, di(trimethylolpropane) tetraacrylate (DTMPTA), trimethylolpropane trimethacrylate, ethoxylated trimethylolpropane triacrylate (ETMPTA), ethoxylated bisphenol diacrylate, hexanediol diacrylate, and mixtures thereof.

22. An electrolyte for activating an electrochemical power source selected from the group consisting of a primary electrochemical cell, a secondary electrochemical cell, and a capacitor, the electrolyte comprising:
a) a small nitrogen onium cation; and
b) an anion selected from the group consisting of closocarborates and halogenated derivatives thereof, closoborates and halogenated derivatives thereof, CF₃SO₃⁻, ClO₄⁻, C(SO₂CF₃)₃⁻, N(SO₂CF₃)₂⁻, O₃SCF₃⁻, C₆F₅SO₃⁻, O₂CCF₃⁻, and those of the following formula:
((R_{f1}SO₂)(R_{f2}SO₂)N)⁻
wherein R_{f1} and R_{f2} are each independently a straight or a branched perhalogenated alkyl group of 1 to 4 carbon atoms, with R_{f1} and R_{f2} having up to 5 carbon atoms, and mixtures thereof.

23. The electrolyte of claim 22 provided in a capacitor of either an electrolytic or an electrolytic/electrochemical hybrid type.

24. The electrolyte of claim 22 wherein the electrolyte is a gel electrolyte.

25. The electrolyte of claim 24 wherein the gel electrolyte includes an unsaturated monomer selected from the group consisting of dipentaerythritol hexaacrylate (DPHA), dipentaerythritol pentaacrylate (DPAA), pentaerythritol tetraacrylate, ethoxylated pentaerythritol tetraacrylate, di(trimethylolpropane) tetraacrylate (DTMPTA), trimethylolpropane trimethacrylate, ethoxylated trimethylolpropane triacrylate (ETMPTA), ethoxylated bisphenol diacrylate, hexanediol diacrylate, and mixtures thereof.

26. A method for providing an ambient temperature molten salt, comprising the steps of:
a) mixing a triethyl amine with a stoichiometric amount of acid;
b) providing lithium bis-trifluoromethanesulfonyl imide;
c) reacting the acidified triethyl amine with the lithium bis-trifluoromethanesulfonyl imide to obtain a mixture containing triethylammonium bis-trifluoromethanesulfonyl imide; and
d) separating the triethylammonium bis-trifluoromethanesulfonyl imide from the mixture.

27. The method of claim 26 including drying the product triethylammonium bis-trifluoromethanesulfonyl imide in a vacuum.
